(19) United States
(12) Patent Application Publication
(10) Pub. No.: US 2008/0274126 A1
(43) Pub. Date: Nov. 6, 2008

(54) Oral Vaccines for Fish

(76) Inventors: Dionisius Elisabeth Antonius Florack, Wageningen (NL); Hendrik Jan Bosch, Wageningen (NL)

Correspondence Address: HOFFMANN & BARON, LLP, 6900 JERICHO TURNPIKE, SYOSSET, NY, 11791, US

(21) Appl. No.: 11/795,937

(22) PCT Filed: Jan. 26, 2006
(86) PCT No.: PCT/NL2006/000044

§ 371 Date: Apr. 14, 2008

(30) Foreign Application Priority Data
Jan. 26, 2005 (EP) 05075203.9

(51) Int. Cl. A61K 39/00; C12P 21/04; C12N 15/11; A61P 37/00; C12N 15/00; C07K 14/00

(52) U.S. Cl. 424/184.1; 435/69.7; 435/69.3; 530/350; 536/23.7; 435/320.1

(57) ABSTRACT

The invention relates to the development, composition and production of mucosal (oral) vaccines for fish. More specifically, the invention relates to protein complexes for the delivery of antigens to and across mucosal surfaces of fish for the induction of an immune response, and to the production of said complexes in a host cell, preferably plants. Provided is the use of a protein complex comprising an antigen of interest fused to the B-subunit of Vibrio cholerae cholera toxin (CT-B), or Escherichia coli heat-labile enterotoxin (LT-B) for the manufacture of an oral fish vaccine. Also provided is fish feed comprising a protein complex of the invention

The invention relates to the development, composition and production of mucosal (oral) vaccines for fish. More specifically, the invention relates to protein complexes for the delivery of antigens to and across mucosal surfaces of fish for the induction of an immune response, and to the production of said complexes in a host cell, preferably plants.

Infectious diseases are the main threat to European and world aquaculture. Intensification of aquaculture has led to an increasing number and frequency of infectious disease outbreaks, resulting in high economical losses and fish suffering. In addition, more fish species are being cultured, each with intrinsic infectious disease risks. Outbreaks often result in high mortality in small as well as in larger fish. Particularly viral diseases are an increasing problem (Leong and Fryer, 1993; Newman, 1993). Examples include Infectious Pancreatic Necrosis (IPN) caused by IPN virus (IPNV) affecting both salmonid and non-salmonid species; Viral Haemorrhagic Septicaemia (VHS) caused by VHS virus (VHSV) and most damaging to farmed rainbow trout (Oncorhynchus mykiss) and Spring Viraemia of Carp (SVC) caused by SVC virus (SVCV) and Koi Herpesvirus (KHV) affecting carp (Cyprinus carpio) but also koi. Other diseases of fish with high probability of occurrence and high economic consequences are Furunculosis, ISA, Hitra disease and SRS in salmon, ERM and Lactococcus in rainbow trout and Vibriosis and Pasteurellosis in sea bass and Vibriosis, Furunculosis and Hitra disease in atlantic cod.

The key to controlling most diseases in fish is identifying the controllable risk factors in disease prevention, rather than putting considerable efforts into eliminating the disease-causing pathogen. It often happens that when a disease occurs, conditions have been created that favour the pathogen over the fish. Whether a fish becomes diseased when a pathogen is present depends on factors that include fish health, water quality and temperature, stocking density, pathogen load, vaccination status, handling practices, uniformity of grade, and proximity of neighboring farms which may experience different disease threats. Of these, vaccination status offers aquaculture producers an effective way to lower both the risk of disease in their fish and their cost of production.

There are three common methods of vaccination: immersion, injection, and oral. These methods vary in terms of ease of administration, cost, stress on the fish, survival rates, dosage control, the amount of labour involved, and the duration of protection. Ultimately, the decision concerning which of these methods to use is based upon a combination of actual and perceived risk, age of the fish, the farmer's own risk-aversion, and return on investment.

It is generally accepted that injectable vaccines provide greater protection than immersion and oral vaccines because they allow for greater dosage control, which results in higher efficacy levels and a longer duration of protection. However, injectable vaccines tend to be more labour intensive, more expensive, and can cause damage to the fish if not administered with care. Furthermore, it precludes its use to small fish and it is expensive. Also, a number of side reactions can occur to either the immunising antigen or to the emulsifying reagent in which it is presented. Hence, injection vaccines if available, are of little use for mass vaccination of small fish under farm conditions.

Immersion vaccination is frequently used in fish farming but has the disadvantages that it is stressful for the fish and not completely protective. Thus, the most attractive method is oral vaccination which is relatively problem free.

Oral vaccine delivery has the major advantage that it enables the farmer to protect juvenile fish as soon as they start to feed. Advantageously, this usually coincides with the moment at which the young fish are most susceptible to attack and colonisation by pathogens. Oral vaccination also enables the farmer to administer booster doses to the fish whenever there is an increased risk of exposure to the pathogen. And last but not least, oral vaccination stimulates immune responses at the portal entrance of many pathogens. The major advantage of oral vaccines is that they enable the farmer to immunise fish with a minimum of stress and handling at mass scale and from the moment immune competent fish start to feed.

The development of practical oral vaccines for aquaculture remains the major elusive goal of the aquaculture industry worldwide. Oral vaccines that can be formulated in fish feed allow the antigen to be administered as soon as immune competent fish start to feed on pelleted feed. Major hurdles which to date have prevented the development of such oral vaccines are 1) the applied antigen (Ag) is often destroyed due to gastric acidity and protease activity present in the intestinal tract; 2) oral tolerance can be evoked and 3) the Ag does not necessarily enters the gut mucosa and consequently an immune response is not initiated.

Destruction of the Ag in the gut can be avoided by Ag encapsulation. For example, oral vaccination with Vibrio anguillarum bacterin antigen encapsulated in alginate microparticles (to protect the vaccine against degradation in the anterior part of the digestive tract) evoked systemic memory and induces mucosal immune responses in fish. (Joosten et al. (1997) Fish and Shellfish Immunology 7: 471). Furthermore, liposomes have attracted considerable interest as carriers and adjuvants for developing oral vaccines. The multilayer phospholipid vesicles protect the entrapped antigen from low pH and enzymatic attack until they reach their target sites (see for example Gregoriadis, Immun. Today 19990; 11:89). Oral vaccination with liposome-encapsulated antigens has been reported in fish. Irie et al. (2003) reported significant increases in anti-BSA antibodies in serum of carp (Cyprinus carpio) upon oral administration of liposome-entrapped bovine serum albumine (BSA) as model antigen.

In general however, the manufacture of these protected oral vaccine formulations is elaborate and expensive. They are therefore not suitable for large scale and cost-effective application, such as in aquaculture.

An object of the present invention is to provide an oral vaccine that allows for the delivery of antigens to fish mucosa which is cost-effective and which can be easily formulated in fish feed.

The invention provides a method for the expression of a protein of interest in a host cell, comprising providing said host cell with a recombinant nucleic acid construct encoding a fusion protein comprising said protein of interest fused to the B subunit of a member of the AB5-class of bacterial toxins, and wherein said protein of interest originates from an organism that has an optimal growth temperature that lies below the optimal growth temperature of said host cell. Also provided is a fusion proteins obtainable by the method, and a protein complex comprising one or more of such fusion proteins. The protein of interest is preferably an antigen of interest, but other types of proteins may of course also be fused to the B-subunit. In particular, it provides the use of a protein complex comprising an antigen of interest fused to the mucosa-binding B-subunit of a B-subunit of a member of the AB5 class of bacterial toxins, such as Vibrio cholerae cholera toxin (CT), or Escherichia coli heat-labile enterotoxin (LT) as an oral fish vaccine. It was surprisingly found that the problems of antigen destruction, antigen uptake as well as oral tolerance in fish can be overcome by the use of functional protein complexes that allow for the delivery of antigens to fish mucosa, promoting binding and uptake of the complex via mucosal cell surface receptors and the induction of immune responses to said antigen. The fusion protein complexes were shown to induce a specific immune response in fish that had been fed with feed pellets containing the protein complex. Surprisingly, antigen protection or encapsulation was not necessary.

The AB5-class of bacterial toxins produced by pathogenic bacteria comprise an A subunit with enzymic activity and a B subunit pentamer responsible for interaction with glycolipid receptors on target eukaryotic cells (Fan, E., E. A. Merritt, C. L. M. J. Verlinde, and W. G. J. Hol. 2000. AB5 toxins: structures and inhibitor design. Curr. Opin. Struct. Biol. 10:680-6861). The class of AB5 toxins may be subdivided into families based on sequence homology and catalytic activity. The cholera toxin family includes in addition to cholera toxin itself the E. coli heat-labile enterotoxins LT and LT-II. The closely-related shiga toxin family comprises a number of toxins from Shigella dysenteriae and the ‘shiga-like’ toxins (also known as verotoxins) from E. coli. The effect of these toxins on human populations ranges from the relatively mild travelers' diarrhea caused by infection with E. coli strains producing LT to the acute and life-threatening diarrhea caused by V cholerae infection and the equally serious hemolytic uremic syndrome (‘hamburger disease’) caused by members of the shiga toxin family.

In mammals, it has been shown that Vibrio cholerae toxin B subunit CT-B and its homologue Escherichia coli heat-labile enterotoxin, LT-B, can be used to target antigens to the immune responsive cells of the mucosa in the digestive tract where they are correctly processed (see e.g. Walker, 1994). The pentameric ring of B subunits binds specific receptors (primarily ganglioside GM1) on the mucosal gut epithelium and can enhance immunogenicity of other antigens when coupled herewith (see Jagusztyn-Krynicka et al., 1993). These findings have led to the development of recombinant enterotoxins as carrier molecules for the presentation of target protein antigens to the immune-responsive cells of the mucosal epithelium in mammals (Aitken and Hirst, 1993; Cardenas and Clements, 1993; Jagusztyn-Krynicka et al., 1993; Khoury and Meinersmann, 1995; Zhang et al., 1995). It has been shown that antigens fused to the carboxyterminus of CT-B or LT-B can elicit humoral and cellular immune responses to said target protein antigens in mammals and birds upon oral administration (Cardenas and Clements, 1993; Jagusztyn-Krynicka et al., 1993; Khoury and Meinersmann, 1995).

Studies on the immunological capacities of the GALT (gut associated lymphoid tissue) of fish have confirmed the existence of a mucosal immune system also in fish (reviewed by Hart et al., 1988). Here, the intestine is also involved in the uptake of orally administered protein antigens (Dalmo et al., 1997; Lamers, 1985; Rombout and van den Berg, 1989; Rombout et al., 1985, 1989) and the production of mucosal immunoglobulins.

It has been shown that anal intubation of carp with either LT-B or LT-B fused to parvo peptide leads to uptake of these peptides in carp gut mucosa and the induction of anti-LT-B and anti-parvo peptide directed humoral immune responses (Companjen et al. Midtlyng P J (ed): Fish Vaccinology. Dev Biol. Basel, Karger, 2005, vol 121, pp 143-150). It will be however understood that immunization of fish by anal intubation is not suitable for large scale application and that oral administration of antigens is obviously the preferred choice. The application of fusion of antigens to enterotoxin B-subunits as oral vaccine for fish as disclosed herein has not been reported before. CT-B subunits have been coupled to liposomes to enhance delivery of liposome-entrapped antigen (BSA) to the intestinal tract in fish (Irie et al., 2003). Liposomes without CT-B were also effective whereas no immune response was observed when fish were orally immunized with BSA-containing unstable liposomes or BSA alone. Thus, according to the teaching of Irie et al. it is of major importance for the induction of serum antibody responses in fish that antigens are protected by their encapsulation in stable liposomes. In marked contrast, the present invention now shows that it is not required to encapsulate or otherwise protect the antigen to induce a humoral immune response in fish following oral vaccination.

Provided herein is the production and use of an antigenic protein complex comprising a B-subunit of an AB5 toxin, fused to an antigen of interest, wherein the protein complex is able to bind to a fish mucosal cell surface receptor, transported across the epithelium and exposed to the fish immune system to result in serum immune responses and protection. The B-subunit may be selected from the group consisting of Heat-labile enterotoxin (LT-B); Shiga en Shiga-like toxin (ST-B); Bordetella pertussis toxin B; Type IIa en b heat-labile enterotoxin, B subunit and cholera toxin B subunit CT-B. In one embodiment the B-subunit is a B-subunit from a member of the CT-family of AB5 toxins, like an LT-B or CT-B subunit, or a B-subunit of a toxin from C. jejuni. The B-subunit can be fused to either the N- or C-terminal end of the protein of interest. Preferably, the B-subunit is fused to the N-terminus of the protein of interest.

A protein complex based on fusion proteins comprising a B-subunit of an AB5 toxin and a protein interest can be prepared using a host cell provided with the suitable nucleic acid construct(s) encoding the components of the complex and allowing expression and assembly of said components into a functional complex. Host cells that can be used for the production of a protein complex include plant cells, fish cells, yeast cells (e.g. Pichia pastoris), algae, for example brown algae such as Egregia enziesii or green algae such as Chlamydomonas rheinhardtii, mammalian cells, fungal cells and insect cells. Suitable fungal cells include Agaricus bisporis, Cantharellus cibarius, Pleurotus spp. and Coprinus spp. Bacterial host cells may be used, for example commensal lactic acid bacteria such as Lactococcus lactis or Lactococcus plantarum. Preferably, a host cell is an edible host cell which does not cause harm upon consumption. In a specific embodiment, a plant can be used to produce an immunogenic protein complex of the present invention. For example, plant cells belonging to monocots or dicots such as corn or rice or potato or tobacco can be used.

Surprisingly, it was observed for some proteins of interest that the coupling to a B-subunit enhances the expression of the protein of interest when expressed as a fusion protein with the B-subunit in a host cell. For example, a nucleic acid sequence encoding a fusion of LT-B and the viral glycoprotein (G) of the fish viruses VHSV or SVCV allowed for optimal transcription and translation initiation in plant host cells. In contrast, the nucleic acid sequence encoding only the viral antigen was not or only very poorly expressed in the host cell. Plant host cells used as recombinant expression system are typically reared in a glasshouse at a temperature between 18 and 30° C. The optimal temperature for viruses that are pathogenic for cold water fish (e.g. VHSV and SVCV) is much lower; it has been observed that the fish viruses VHSV and SVCV flourish best between 8 and 14° C. Furthermore, no pathogenicity of these viruses is observed at higher temperatures. This may indicate that recombinant expression of the viral G proteins alone in a plant host cell is hampered by the fact that the plants are grown at a temperature (18-30°) well above the temperature at which the antigens are normally expressed (8-14° C.). Both viral glycoproteins are multimeric proteins and it is conceivable that their folding and multimerization cannot take place properly at supra-optimal temperatures such that they are more susceptible for proteolytic degradation. Without wishing to be bound by theory, it is proposed that the coupling of the viral antigens to AB5 B-subunits that are capable of forming pentameric complexes when expressed in plants enhances the stability and expression of the antigen. The invention therefore provides a method for the expression of a protein of interest in a (plant) host cell wherein said protein originates from an organism that has an optimal growth temperature that lies below the optimal growth temperature of said host cell, wherein said protein is expressed as a fusion protein with a B-subunit of member of the AB5-class of bacterial toxins, for instance Vibrio cholerae cholera toxin (CT) or Escherichia coli heat-labile enterotoxin (LT). Said host cell is for example a plant cell and said protein of interest is for example derived from a marine animal (e.g. fish) or an organism pathogenic to a marine animal.

A further embodiment relates to a nucleic acid construct for use in a method of the invention. The construct encodes a fusion protein comprising a protein of interest which originates from a virus or micro-organism pathogenic to fish fused to the B subunit of a member of the AB5-class of bacterial toxins, preferably the B-subunit of Vibrio cholerae cholera toxin (CT-B) or Escherichia coli heat-labile enterotoxin (LT-B). For example, the encoded protein of interest originates from a fish pathogenic, preferably selected from the group consisting of infectious pancreatic necrosis virus (IPNV), striped jack nervous necrosis virus (SJNNV), infectious haematopoietic necrosis virus (IHNV), viral haemorrhagic septicaemia virus (VHSV), Pancreas Disease virus (SPDV), infectious salmon anaemia virus (ISAV), Spring Viraemia of Carp virus (SVCV), Koi Herpesvirus (KHV), Flexibacter columnaris, Edwardsialla ictaluri, E. tarda, Piscirickettsia salmonis, Vibrio spp and Aeromonas spp., Yersinia ruckeri, Pasturella piscicida and Renibacterium salmoninarum. In one aspect, the nucleic acid construct of the invention encodes a B-subunit of a AB5 toxin fused to an antigen of interest selected from the group consisting of the VP2-protein of IPNV, the glycoprotein of VHSV (VHSV-G) and the glycoprotein of SVCV (SVCV-G). Also provided is an expression vector, preferably a plant expression vector, comprising a nucleic acid construct according to the invention. The expression vector is suitably used to for the recombinant expression of the construct in a (plant) host cell. Standard recombinant DNA technology can be used to prepare the required constructs. The construct can be introduced in the host cell by various conventional techniques, including transfection, electroporation and Agrobacterium-mediated gene transfer. A person skilled in the art will be able to choose the most suitable technique for a particular host cell.

The method of the invention is advantageously used for the expression of a protein of interest that in its native form exists as a multimer, for example as a dimer or a trimer. Following expression in the (plant) host cell, the fusion protein can be used as such (e.g. as an antigenic protein complex of the invention in an oral vaccine composition). Alternatively, the fusion protein can be further processed to remove the B-subunit and release the protein of interest. Processing can be performed enzymatically, e.g. using a protease like trypsin, or chemically.

Also provided is a fusion protein obtainable by a method according to the invention, and a protein complex comprising one or more fusion proteins.

Furthermore, the invention provides a vaccine, in particular an oral fish vaccine, comprising a fusion protein of the invention or a protein complex of the invention. In a preferred embodiment of the present invention, the fusion protein comprises a B-subunit fused to an antigen of interest selected from the groups consisting of a viral, bacterial or microbial surface antigen of a fish pathogen. Examples of a virus or micro-organism pathogenic to fish include infectious pancreatic necrosis virus (IPNV), striped jack nervous necrosis virus (SJNNV), infectious haematopoietic necrosis virus (IHNV), viral haemorrhagic septicaemia virus (VHSV), Pancreas Disease virus (SPDV), infectious salmon anaemia virus (ISAV), Spring Viraemia of Carp virus (SVCV), Koi Herpesvirus (KHV), Flexibacter columnaris, Edwardsialla ictaluri, E. tarda, Piscirickettsia salmonis, Vibrio spp and Aeromonas spp., Yersinia ruckeri, Pasturella piscicida and Renibacterium salmoninarum. Of course, antigens that have been shown to be protective antigens in non-oral vaccine formulations in fish are of particular interest for the present invention. Known protective antigens that are suitably used in an oral vaccine formulation according to the invention include the G protein of Viral Haemorrhagic Septicemia virus (VHSV; Lorenzen et al., 1998).

Furthermore the invention provides a method for immunizing fish, comprising the oral administration of a vaccine composition comprising a fusion protein or protein complex of the invention. Preferably, oral administration of the vaccine composition comprises feeding fish with fish feed containing said fusion protein.

A functional protein complex of the invention has a pentameric structure required for interaction with mucosal cell surface receptors. In one embodiment, it is a homopentamer of five identical antigen-B-subunit fusion proteins. One B subunit can be fused to a single antigen or to multiple copies of that antigen. It is also possible to incorporate more than one type of antigen in a protein complex of the invention. For example, a tandem repeat of different types antigens can be fused to one B-subunit. In an alternative embodiment, the antigenic protein complex is a heteropentameric complex composed of five B-subunits fused to different antigens. For example, two subunits of the complex are fused to antigen A and three subunits of the complex are fused to antigen B. Other combinations are of course also possible. Furthermore, not all B-subunits need to be fused to an antigen of interest. Use of protein complexes consisting of at least one ‘free’ or “unaltered” B-subunit and at least one fused B-subunit as an oral fish vaccine is also encompassed. In fact, it is believed that for certain antigens, in particular large antigens, it is preferred that not all subunits of the pentamer are loaded with antigen because the antigen may otherwise interfere with the formation of a functional pentamer (see application PCT/NL2004/000708).

In a further aspect, a protein complex comprising an antigen of interest as disclosed herein is advantageously formulated into an oral vaccine composition that can be administered to fish in a non-labour-intensive manner, e.g. being part of fish feed particles or pellets, and causes no stress to the fish, in complete contrast to conventional vaccine administration by parenteral injection. Herewith, the invention provides a fish feed composition comprising a protein complex comprising a protein (e.g. antigen) of interest fused to the B-subunit of an AB5 bacterial toxin. In one embodiment, a fish feed composition comprises a protein complex comprising an antigen of interest fused to the B-subunit of Vibrio cholerae cholera toxin (CT-B), or to the B-subunit of Escherichia coli heat-labile enterotoxin (LT-B). A fish feed composition of the invention for instance comprises feed pellets or particles to which a protein complex of the invention has been added either during or after manufacture of the fish feed. Dry pellet fish feed is getting popular among aquaculture industry in the last few years. Pelleted commercial fish feeds are available in a variety of pellet sizes. The main ingredient of most types of dry pellet fish feed is fish meal, protein from other animal or plant origin, fish oil or other kinds of lipids, vitamin premix, minerals and binders in accordance with the nutritional requirement of the target cultured species. The combined ingredients are usually extruded into pellets of different sizes and densities to suit the feeding behaviour of different types of cultured fish. The choice of the size of pellet to feed is typically based on the size of the fish. In one embodiment, fish pellets prepared in the conventional manner are coated with a composition comprising an antigenic protein complex of the invention. Preferably however, pellets are prepared from a mixture of the conventional pellet ingredients and a protein complex of the invention. The protein complex can be added to fish feed in a crude or a (partially) purified form. For example, if an edible host cell is used for the production of a protein complex, the host cells expressing the components of the complex can be used as such in fish feed. In a specific embodiment, feed pellets are provided that contain a certain amount (e.g. 20% by weight) of freeze-dried potato tuber material obtained from transgenic potato host cells expressing LT-B-antigen fusion protein. This corresponded to approximately 4-5 micrograms of fusion protein per gram of food. Feeding these pellets to fish resulted in a systemic humoral immune response to the antigen.

The invention is illustrated by the Examples below using one type of AB5 toxins (LT). However, the skilled person will understand that other types of B-subunits can be used interchangeably when practicing the invention.

### LEGENDS

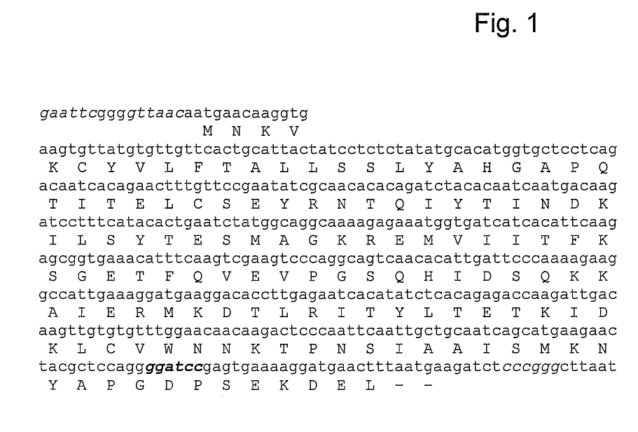

FIG. 1. Gene sequence synthetic gene for LT-B as present in pLANTIGEN4. In italics are EcoRI, HpaI, BamHI and SmaI sites used for cloning. Putative translation is given in single letter amino acid abbreviation.

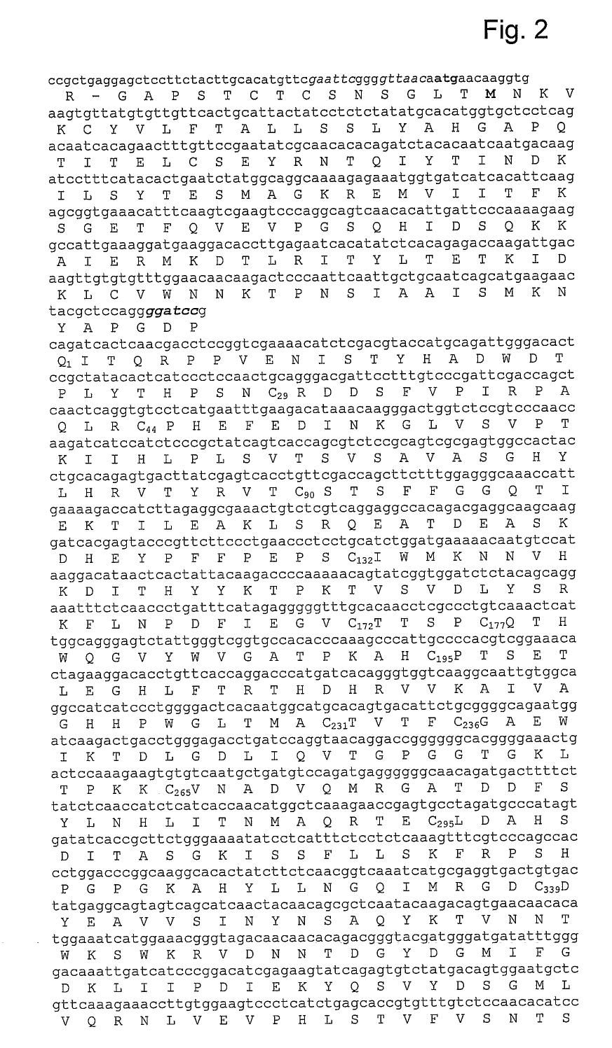

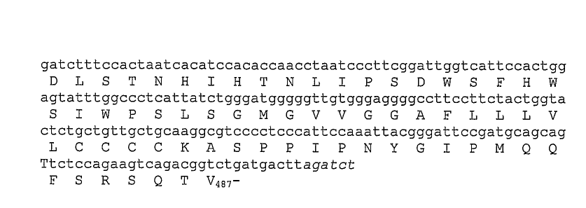

FIG. 2. Gene sequence fusion synthetic gene for LT-B and VHSV G as present in pLANTIGEN24. Putative translation is given in single letter amino acid abbreviation.

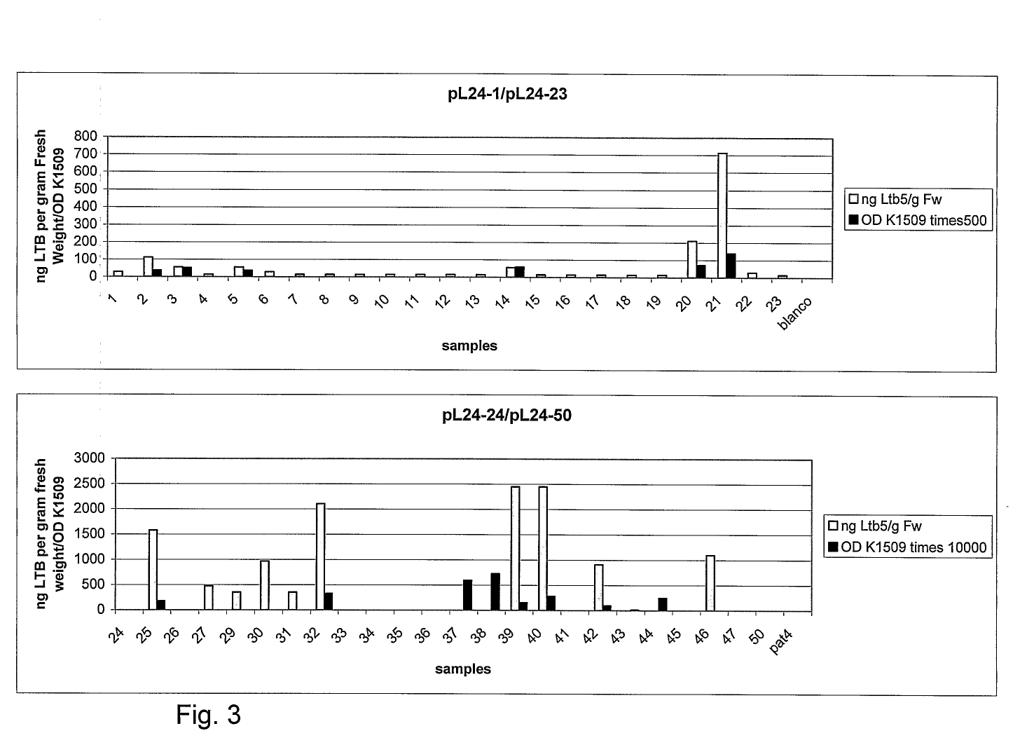

FIG. 3. Results of GM1 ELISA (light bars) and modified GM1 ELISA using K1509 polyclonal antibody against VHSV G (dark bars), for all pLANTIGEN24 transgenic tuber plant extracts. Panel A shows the results for plants 1 to 23. Panel B shows the results for plants 24 to 50. Five micrograms of total potato tuber extract was loaded onto GM1 plate. Detection was with VD12 (Lauterslager et al., 2001) for the presence of LT-B5 pentamers (Ltb5) and K1509 for the presence of VHSV G.

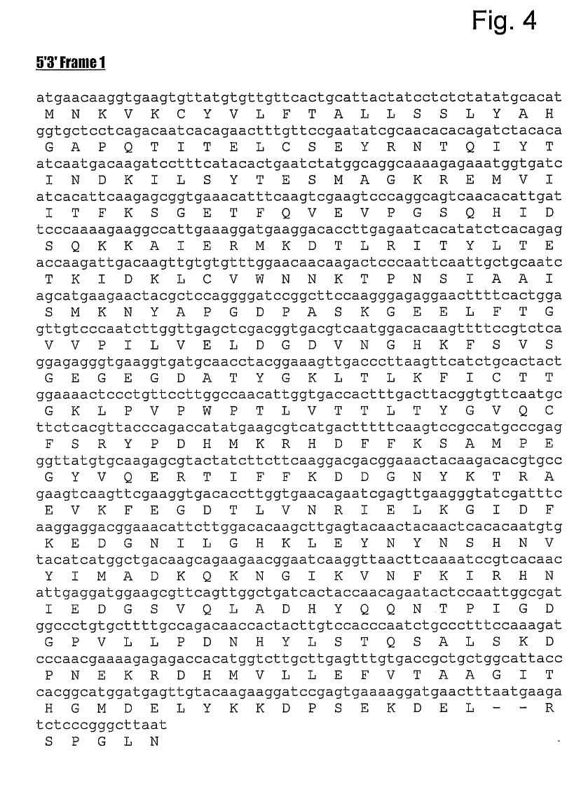

FIG. 4. Gene sequence fusion synthetic gene for LT-B and GFP sequence as present in pLANTIGEN20. Putative translation is given in single letter amino acid abbreviation.

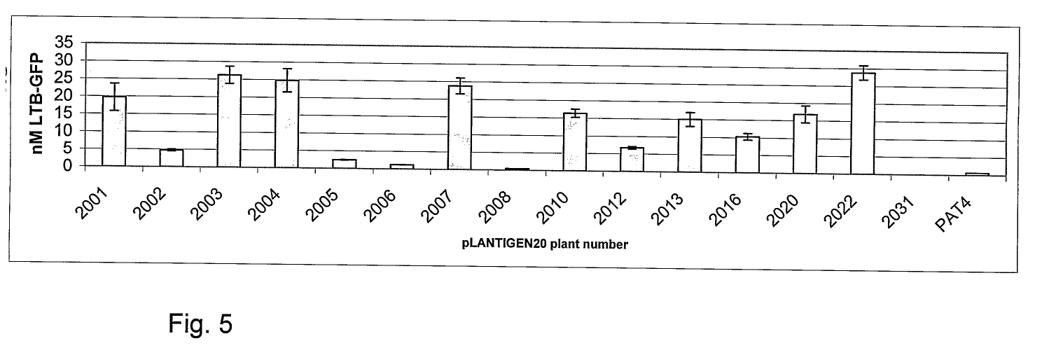

FIG. 5. Results of GM1 ELISA for selected pLANTIGEN20 (LT-B-GFP) transgenic tuber plant extracts. Detection was with VD12 (Lauterslager et al., 2001) for the presence of LT-B5 pentamers. PAT4, negative control. Concentration is given in nM.

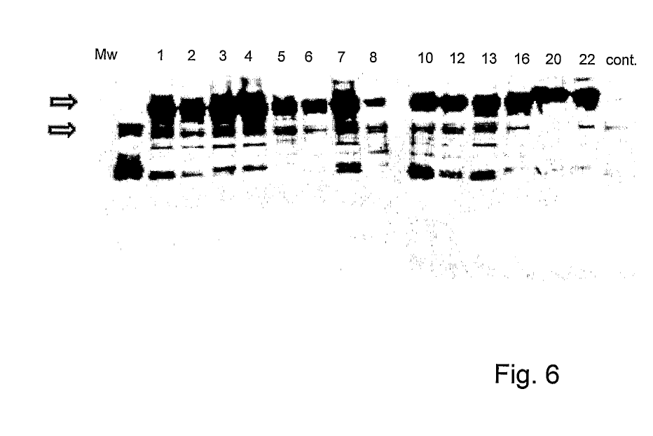

FIG. 6. Western blot analysis of pLANTIGEN20 (LT-B-GFP) transgenic tuber extracts under semi-native conditions. Numbers above the lanes indicate the individual plant number (lanes 3 to 16). Lane 2, pLANTIGEN4 (LT-B) positive control. Cont. Refers to extract of a PAT4 (empty vector) negative control. Blots were probed with the monoclonal antibody VD12 to detect pentameric complexes of LT-B. The upper arrow indicates the position of a high molecular weight complex of the LT-B-GFP complex whereas the lower arrow indicates the LT-B complex of appr. 60 kDa.

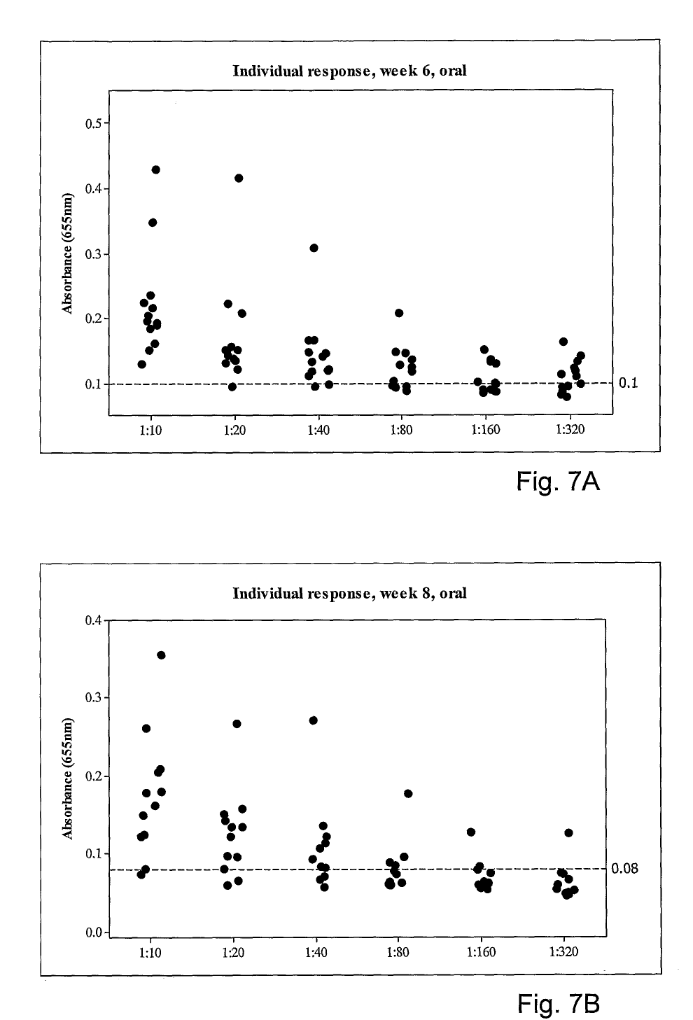

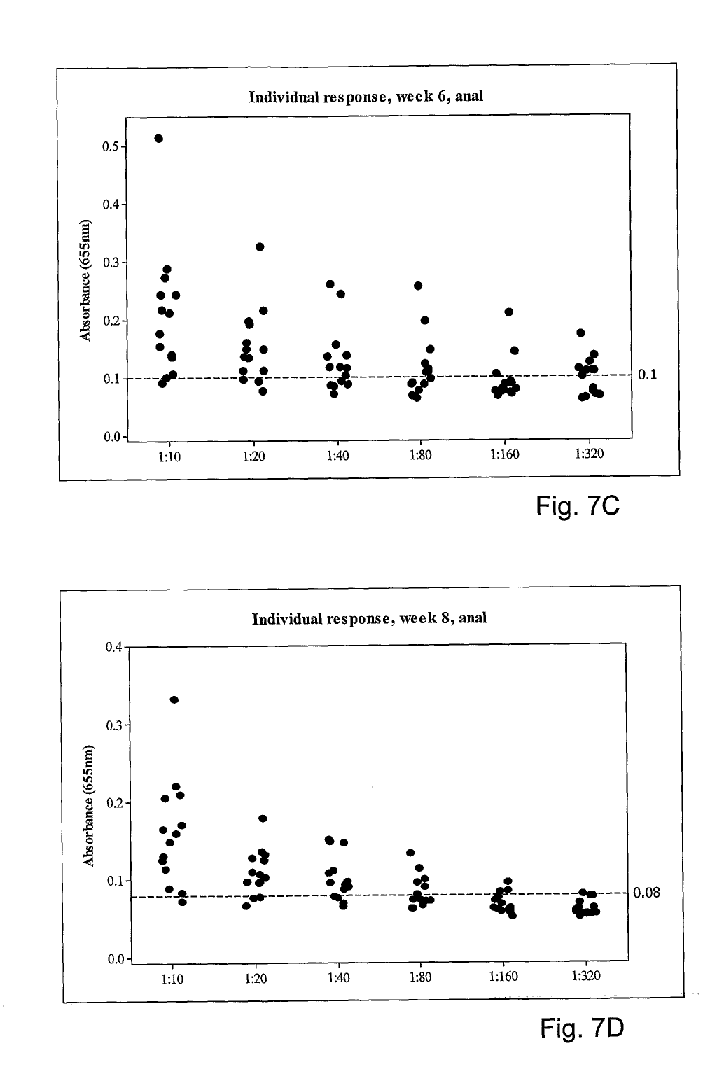

FIG. 7. Results of oral (panels A en B) and anal (panels C and D) immunization experiments of trout with LT-B-GFP protein complex produced in potato tubers. Six (panels A and C) and eight weeks (panels B and D) after immunization sera was collected. Various dilution of the sera (1:10 to 1:320) were assayed for the presence of anti-GFP antibodies by an Elisa assay using GFP-coated plates, mouse anti-trout and horseradish-peroxidase coupled goat anti-mouse antibodies. The absorbance at 655 nm is reflective of the amount of complex formed between GFP and anti-GFP antibodies in the serum (for details see Example 3). Test sera are considered positive if the absorbance was larger than two-fold the negative control sera absorbance (indicated by dotted lines).

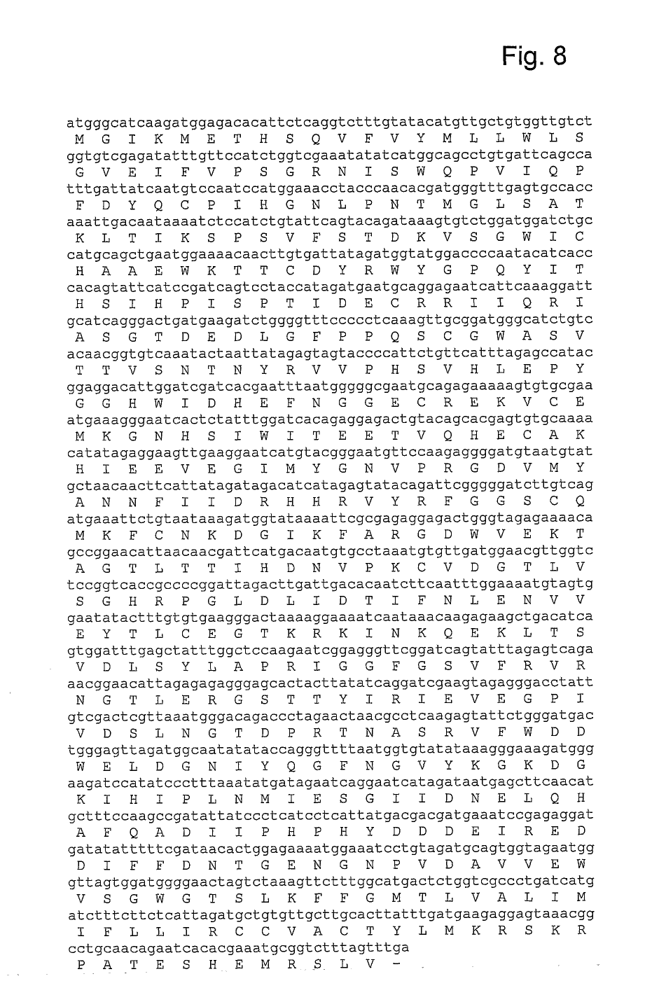

FIG. 8. Gene sequence of SVCV-G as present in pLANTIGEN25. Putative translation is given in single letter amino acid abbreviation.

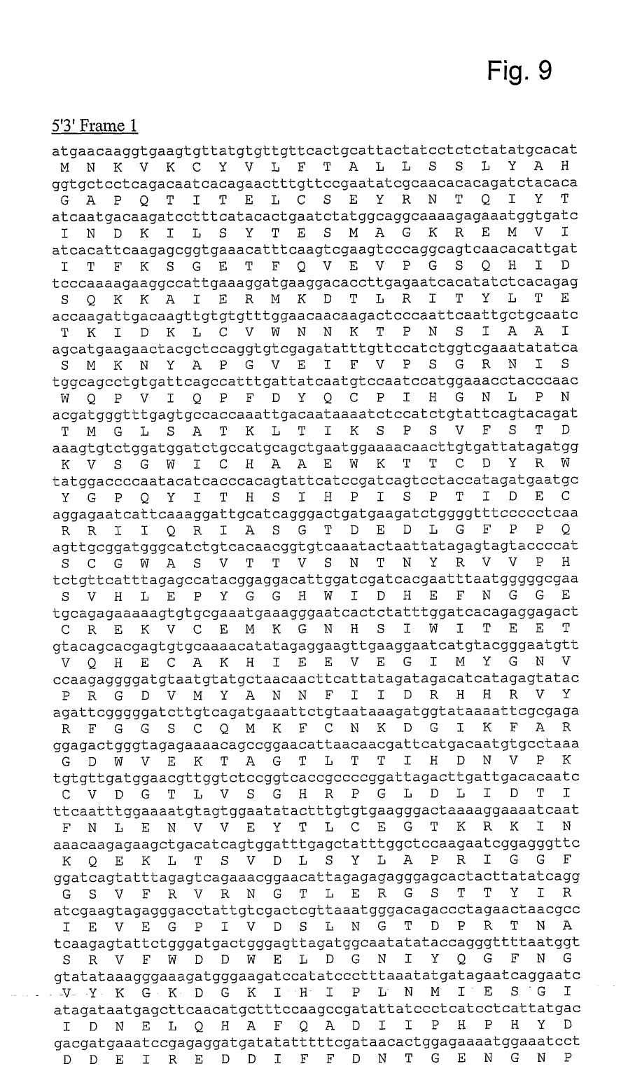

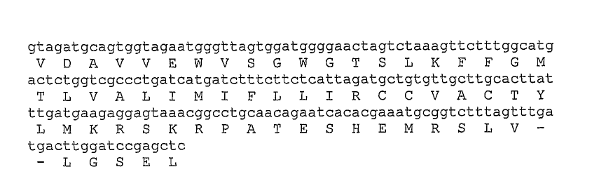

FIG. 9. Gene sequence fusion synthetic gene for LT-B and sequence SVCV-G as present in the fusion construct pLANTIGEN27. Putative translation is given in single letter amino acid abbreviation.

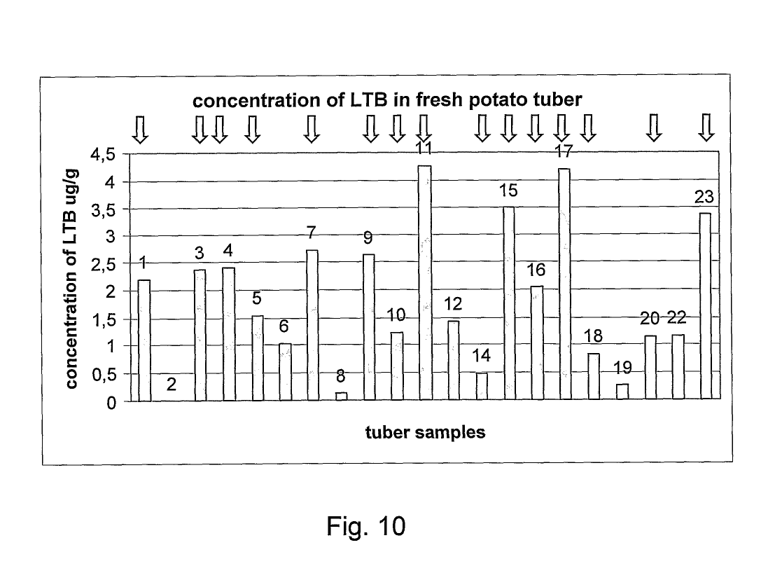

FIG. 10. Analysis of functional LT-B pentamers in tuber extracts of transgenic plants transformed with a mixture of pL4 (encoding LT-B) and pL27 (encoding the LT-B-SVCV-G fusion protein). Pentamer formation was determined by GM1 ELISA. To analyse the presence of SVCV G protein in the pentameric complexes, a modified GM1 ELISA was performed in which the detection of bound complexes with VD12 (specific for LT-B5) was now done with monoclonal antibody 2C1/3C9 specific for SVCV G. Tuber samples that reacted positively in this modified GM1 ELISA are indicated in with an arrow.

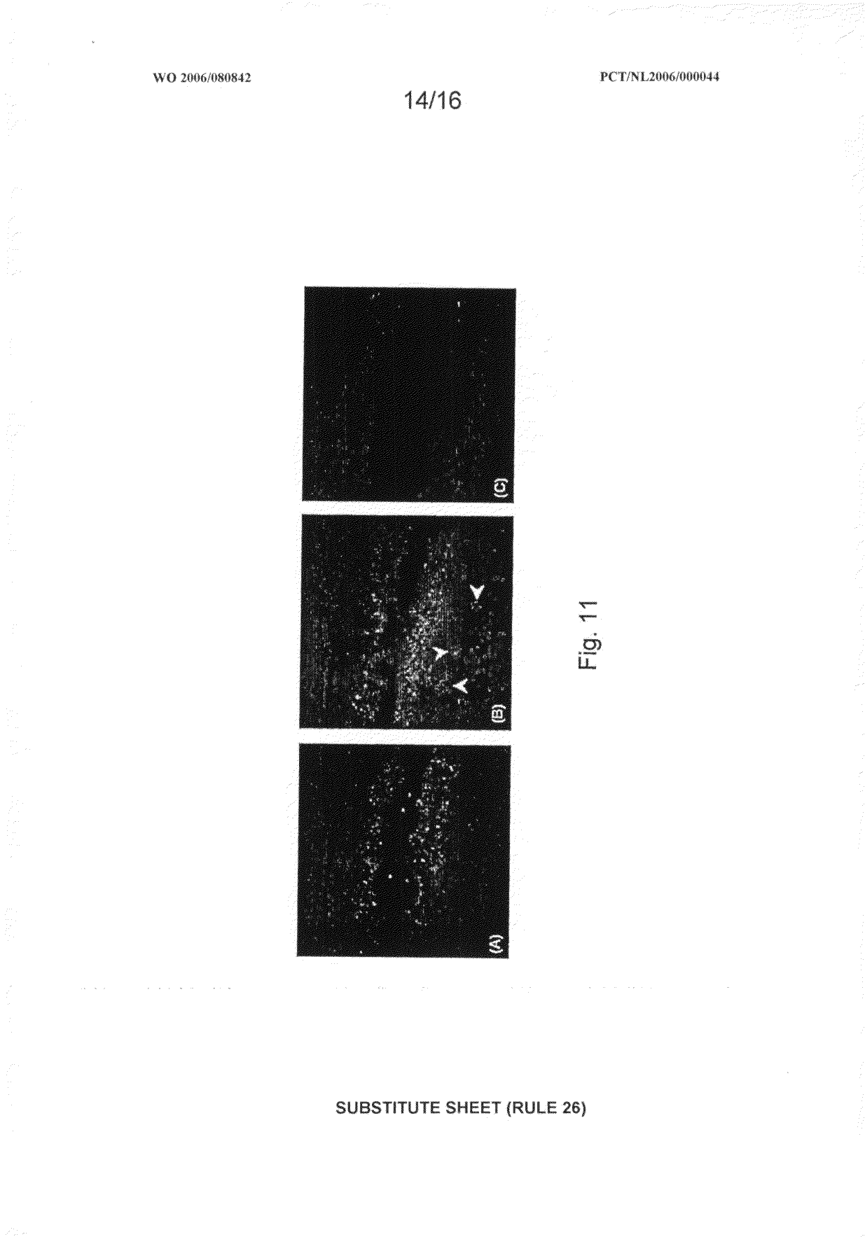

FIG. 11. LTB facilitates a better uptake of GFP in carp gut mucosa. Sections of carp gut intubated with (panel A) GFP, (panel B) LTB-GFP containing potato suspension and (panel C) control potato suspension and incubated for 6 hours. Note the large macrophage-like cells in the LTB-GFP intubated gut (arrowheads).

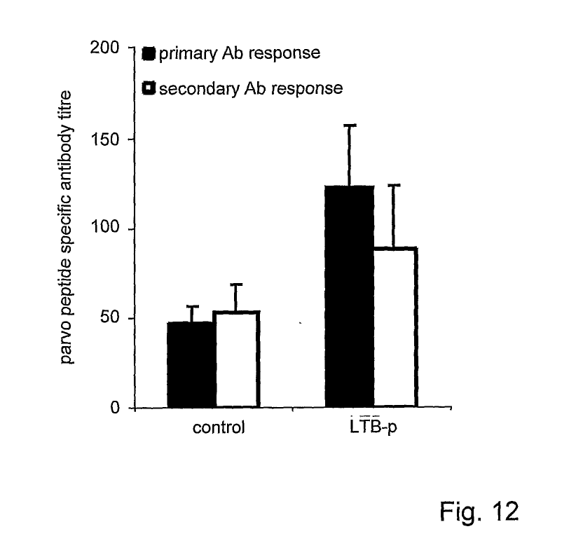

FIG. 12. Systemic parvo peptide-directed humoral immune responses are induced upon anal intubation. Parvo peptide-specific antibody responses were measured in primary and secondary immune serum of fish anally immunised with LTB-p (LTB-parvo peptide). The parvo peptide specific antibody responses were measured by ELISA. The mean antibody titre ±SEM of 3 fish is shown.

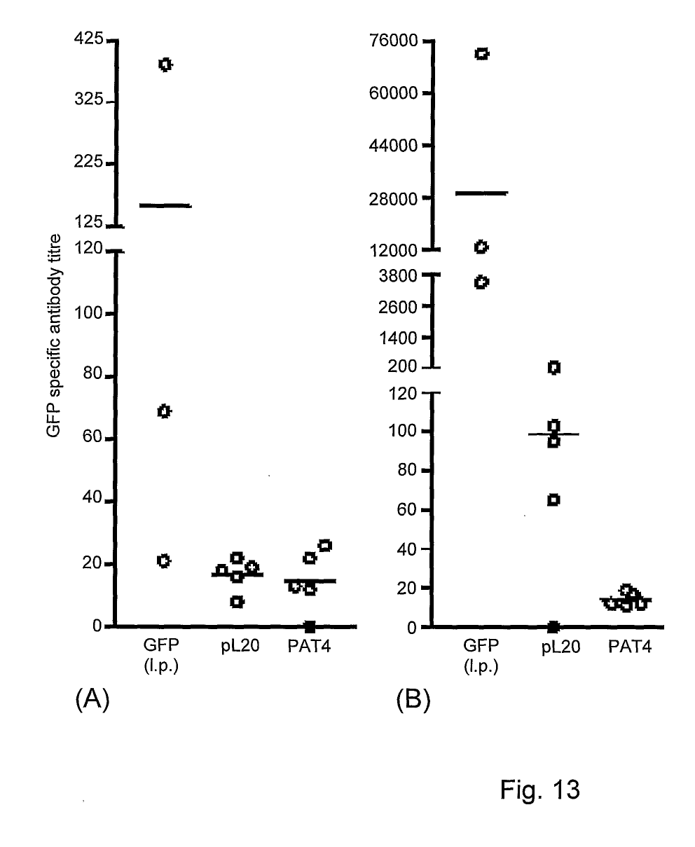

FIG. 13. Oral immunisation with pL20 (LTB-GFP) treated food pellets results in an anti-GFP response. Carp were orally immunised by feeding with pL20 or PAT4 (empty vector control) mixed food pellets or pL20 or PAT4 coated food pellets. Anti-GFP responses in (A) primary immune serum and (B) secondary immune serum are shown. Anti-GFP responses were measured by ELISA. GFP i.p.: anti-GFP responses in serum of fish i.p. immunised with GFP in IFA.

### EXAMPLE 1

### Oral Vaccine for Infectious Pancreatic Necrosis

Introduction. Infectious Pancreatic Necrosis (IPN) is a viral disease caused by IPN virus (IPNV), the prototype virus of the family Birnaviridae. It is an important viral disease that affects both salmonid and non-salmonid finfish species and is distributed worldwide. Only specific data for Norwegian Atlantic salmon industry are available where estimated losses due to IPN are estimated to be approximately 60 million ECU annually (Christie, 1997). The world-wide enzootic distribution of IPNV, and other birnaviruses, suggests that their successful control will be crucial for future cultivation of both new and existing fish species. IPNV possesses a double-stranded bi-segmented RNA genome. Its viral particles consist of an unenveloped icosahedral 60 nm capsid. The viral genome is transcribed into two non-polyadenylated sequences corresponding in size to the A and B segments of the genome. The sequence of the viral A segment encodes an approximately 100 kDa polyprotein which is cleaved to produce, in order from the amino terminal end, the major virion protein VP2, and the minor structural proteins VP4 and VP3. The major capsid protein VP2 is produced from a precursor protein pVP2 and is then assembled to form the capsid. VP2 protein was shown to be protective and is the sole antigen present in a commercially available injection vaccine.

Gene construct. A genetic fusion of a synthetic gene for LT-B, optimized for expression in potato and other Solanaceae, and the coding sequence for the VP2 major capsid protein of IPNV can be made as follows: the gene coding for the major capsid protein VP2 of IPNV is adapted for cloning into the unique BamHI site of pLANTIGEN4 in frame at the C terminus of the synthetic gene for LT-B (Lauterslager et al., 2001; FIG. 1) by PCR amplification of a template resembling the gene sequence of genbank accession U48225 using primers IPNV for 5′-caggggatcccatgaacacaaacaaggcaaccgc-3′ and IPNVrev 5′-aaaaacccgggagatctcattacacctcagcgttgtctccgc-3′. The respective BamHI/SmaI fragment can be cloned under control of the patatin promoter in pLANTIGEN4 (Lauterslager et al., 2001) to generate pLANTIGEN32 (LT-B-IPNV VP2). Transgenic plants can be made as described in Lauterslager et al. (2001) and plants can be selected by GM1 ELISA of potato tuber extracts.

### EXAMPLE 2

### Oral Vaccine for VHSV

Gene constructs and transformation. To enable recombinant VHSV G protein synthesis in transgenic plants, gene construct pLANTIGEN21 was made and transformed into potato. PLANTIGEN21 was made as follows: unique SalI and BglII sites were introduced at the N- and C-terminus of the mature G protein coding sequence of VHSV G by amplification of pcDNA3vhsG (McLauchlan et al., 2003) with oligonucleotides VHSV G1, 5′-tctggtgtcgaccagatcactcaacgacctccgg-3′ and VHSV G3,5′-gatcgaagatctaagtcatcagaccgtctgacttctg-3′ by PCR under optimum conditions using a proof-reading Pwo polymerase for amplification. An NcoI/SalI fragment comprising a signal peptide for secretion (Van Engelen et al., 1994) was ligated to the respective SalI/BglII digested PCR fragment and placed under control of the patatin class I promoter and nopaline synthase terminator by cloning the resulting NcoI/BglII fragment in the NcoI/BamHI sites of pLANTIGEN4 (Lauterslager et al., 2001; FIG. 1) to generate pLANTIGEN21. The latter was transformed to potato as described (Lauterslager et al., 2001) and 58 transgenic plants were regenerated and grown to maturity in the greenhouse.

Accordingly, a genetic fusion of LT-B and the G protein of VHSV was constructed by introducing an unique BamHI site at the N terminus of VHSV G protein coding sequence in pLANTIGEN21, and an unique SmaI site at the C-terminus, by PCR amplification using primers G-F, 5′-caggggatcccagatcac-3′ and Glong-R-SmaI, 5′-aaaaacccgggagatctcattaaagttc-3′. The resulting BamHI/SmaI fragment was cloned in frame in the unique BamHI site at the C-terminus of LT-B coding sequence in pLANTIGEN4 (Lauterslager et al., 2001) generating pLANTIGEN24 (FIG. 2). The latter was transformed into potato as described and 47 independent transgenic plants were regenerated and grown to maturity in the greenhouse.

Analysis of expression. Tuber extracts of pLANTIGEN22 tubers were analysed for the presence of VHSV′G protein by sandwich ELISA using monoclonal antibodies 3F1A2, IP1H3 and 3F1H10 (Cupit et al., 2001) as described (Lorenzen et al., 2000). None of the extracts of transgenic plants showed expression of the G protein. Potato tuber extracts of all pLANTIGEN24 tubers were analysed by GM1-ELISA for the presence of LT-B5 pentamers (Lauterslager et al., 2001). To enable detection of VHSV G protein, a modified GM1-ELISA was performed by incubating microplates coated with purified bovine brain GM1 ganglioside with potato tuber extracts, followed by incubation with K1509 a polyclonal antibody against VHSV G recognizing viral VHSV G protein. Using this assay, the presence of VHSV G protein in a complex with pentameric LT-B5 can be established. The results of both GM1 ELISA and the latter modified GM1 ELISA are summarized in FIG. 3 for all pLANTIGEN24 plants. As can be seen from FIG. 3, by making a fusion protein comprising LT-B and the VHSV G protein, GM1 binding LT-B-VHSV G protein complexes can be established in transgenic potato tubers. There is a strong correlation between the level of GM1 binding activity and recognition by the polyclonal K1509 antibody of GM1 bound complexes, as expected. At least 19 transgenic plants showed expression of GM1 binding LT-B above background (approximately 1 nanogram/gram fresh weight). More than half of these also were positive for VHSV G protein whereas none of the pLANTIGEN22 was. Levels were up to 2.5 micrograms of LT-B5 per gram fresh weight tuber. Selected plants pL2420 (i.e. plant number 20 of the group of plants transgenic for pL24) and pL2421 were also positive in a sandwich ELISA with the two monoclonal antibodies 3F1A2 and IP1H3, suggesting proper folding of at least the two conformational epitopes that are recognized by these two mAbs (Lorenzen et al., 2000).

Immunogenicity and challenge experiments. Extracts and freeze dried tuber material of selected plants including pL2421, are used in a vaccination trial and challenge experiment. Rainbow trout fingerlings of approximately 4 grams (120 per group) are immunized intraperitoneally by injection of 50 microliters of a 10% pL2421 extract mixed with Freunds incomplete adjuvant, intramuscularly by injection of 25 microliters of a 10% homogenate in phosphate-buffered saline (PBS) or orally by application of 100 microliter of 5% homogenate in fish oil, twice and challenged 6 weeks post vaccination with VHSV. Sera are collected 6 weeks post vaccination to study antibody responses. Challenges are as described (McLauchlan et al., 2003). Alternatively, freeze dried tuber material of selected transgenic plants expression LT-B complexes and positive for VHSV G, including pL2420, pL2421, pL2439 and pL2440, are incorporated into a standard fish meal and resulting pellets are used for oral in feed immunization.

### EXAMPLE 3

### Oral and Anal Intubation of Trout with LT-B-GFP

Gene construct LT-B-GFP. A gene fusion of LT-B and green fluorescent protein (GFP) was made as follows: a unique BamHI site was introduced in the coding sequence of a GFP sequence by PCR using oligonucleotides GFPFw 5′-aggggatccggettccaagggagaggaac-3′ and GFPRev 5′-ctcggatccttcttgtacaactcatccatgcc-3′. The resulting BamHI fragment was cloned in the unique BamHI site at the C-terminus of a synthetic gene for LT-B in pLANTIGEN4 (Lauterslager et al., 2001; FIG. 1) to generate a translational fusion LT-B-GFP named pLANTIGEN20 (FIG. 4). The latter binary vector was introduced in Agrobacterium tumefaciens strain Agl0 and used for transformation of potato cultivar Desiree as described before (Lauterslager et al., 2001).

Tuber analysis. Thirty-one (31) transgenic plants were generated and grown to maturity in the greenhouse. Tuber tissue slice analysis for green fluorescence at 480 nm indicated that almost all were positive for GFP. Half of the transgenic tubers were analysed by GM1-ELISA for the presence of LT-B5 pentameric complexes and results are summarized in FIG. 5. Western blotting under semi-native conditions and using the LT-B5 conformational monoclonal antibody VD 12 for detection of pentameric complexes, indicated the presence of high molecular weight complexes in transgenic plants positive in GM1 ELISA (FIG. 6). From FIG. 6 it can be seen that all transgenic pL20 plants accumulated high molecular weight complexes that are recognized by VD12 (left upper arrow) whereas for control pL4 plant expressing the synthetic gene for LT-B, an approximately 60 kDa complex can be visualized (lower arrow). Plant pL2003 was selected which showed expression of LT-B-GFP pentameric complexes at approximately 25 nM scale which is equivalent to 5.3 micrograms of complex per gram fresh weight (FIG. 5). pL2003 was further grown in the greenhouse for bulk tuber production. Tubers were harvested, peeled and freeze-dried. Fish were either immunized with freeze-dried pL2003 material or with fish feed comprising 20% of pL2003 freeze-dried tuber material.

Immunization experiments rainbow trout. Rainbow trout (mean weight 84.9 g) were immunised by oral or anal intubation with selected freeze-dried pL2003 homogenized potato tuber material comprising approximately 25 micrograms of LT-B-GFP per gram dry weight. Prior to immunisation, fish were starved for 24 h. Potato tuber material expressing LT-B-GFP was passed through a 100 μm mesh utilising a pestle and then suspended in PBS (0.15M, pH 7.2) to a final concentration of 200 mg DW/ml which approximates 5 micrograms/ml of LT-B-GFP. Trout were anaesthetised by exposure to benzocaine (50 mg/l) and were either orally, 200 microliter (effective concentration 1 microgram LT-B-GFP) or anally immunised with 100 μl of re-suspended tuber material (effective concentration 500 nanograms LT-B-GFP) via a short section of plastic tubing attached to a 1 ml syringe. Groups consisted of 15 fish per delivery route and fish were identified by marking them sub-cutaneously with alcian blue dye. Fish were then returned to holding tanks, 1 m in diameter, 340 l in volume 15 l/min. Temperature for the experimental period ranged from 8.7 to 14.2° C. Fish were bled at weeks 6 and 8 post-intubation. Blood was collected, allowed to clot overnight at 4° C. and then centrifuged at 3500 rpm for 15 mins and the sera collected, aliquoted and frozen at −80° C. until assayed. Purified recombinant GFP expressed in E. coli and purified was used for coating. Ninety-six well ELISA plates (Immulon 4, Dynex) were coated with E. coli expressed GFP (50 μl 10 μg/ml in carbonate-bicarbonate buffer 0.05 M, pH 9.6) and left overnight at 4° C. Plates were washed with PBS containing 0.05% Tween 20 (PBST, 3×2 min) and blocked with 5% dried non-fat skimmed milk in PBST (100 μl/well) for 2 h at 37° C. Plates were washed as before and frozen at −20° C. Sera were tested as follows. Ninety microlitres of PBS containing 1% bovine serum albumin was added to each well. Ten microlitres of test fish sera was added to column 1 and 7 and doubly diluted across the plate to columns 6 and 12 respectively, giving a dilution range of 1:10 to 1:320. Sera were incubated for 2 h at room temperature before washing as before. Subsequently, 50 μl I-14 (monoclonal to trout Ig, 1:2 PBST) was added and incubated for 1 h at 37° C. Plates were washed as before, and 50 μl goat anti-mouse horseradish peroxidase (HRP) conjugate (Sigma) was added (1:70,000 PBST) and incubated for 1 h at 37° C. Plates were then washed 2×2 min PBST followed by 1×2 min PBS and 100 μl tetramethylbenzidine (TMB) substrate (Sigma) was added and plates incubated for 30 min in dark. Plates were then read at 655 nm and the absorbance recorded. Each plate consisted of test sera, known positive sera (from fish i.p. injected with GFP in Freunds complete adjuvant) and negative sera. In all plates, positive sera exhibited absorbencies that were approximately 10 fold higher than the negative sera. Test sera were considered positive at each dilution if the absorbance was greater than 2× the negative control sera absorbance. The quoted titre is therefore the last one that was 2× that of the negative control. Results are summarized in FIG. 7. FIG. 7 shows that 6 weeks post oral immunization (panel A) all fish have immune responses above background at 1:10 dilution and more than half of the fish also at 1:320 dilution. Antibody responses persist at 8 weeks post oral immunization (panel B) whereas antibody responses upon anal intubation (panels C and D) slightly decrease in time (8 weeks, panel D compared to 6 weeks post immunization, panel C).

### EXAMPLE 4

### Oral Vaccine for SVC in Carp

Gene constructs and transformation. To enable recombinant SVCV G protein synthesis in transgenic plants, the gene construct pLANTIGEN25 was made and transformed into potato. PLANTIGEN25 was made as follows: unique XhoI and BamHI sites were introduced at the N- and C-terminus of the mature G protein coding sequence of SVCV G by amplification of pcDNA3-svcG-539 with oligonucleotides SVCVG1, 5′-tctggtctcgagatccccatatttgttccatc-3′ and SVCVG2,5′-gatcgaggatccaagtcatcaaactaaagaccgcatttcg-3′ by PCR under optimum conditions using a proof-reading Pfu polymerase for amplification. An NcoI/XhoI fragment comprising a signal peptide for secretion (Van Engelen et al., 1994) was ligated to the respective XhoI/BamHI digested PCR fragment and placed under control of the patatin class I promoter and nopaline synthase terminator by cloning the resulting NcoI/BamHI fragment in the NcoI/BamHI sites of pLANTIGEN4 (Lauterslager et al., 2001; FIG. 1) to generate pLANTIGEN25 (FIG. 8). The latter was transformed to potato as described (Lauterslager et al., 2001) and 47 transgenic plants were regenerated and grown to maturity in the greenhouse. In addition, a genetic fusion of LT-B and the G protein of SVCV was constructed as follows: unique NcoI and SalI sites were introduced in the synthetic gene for LT-B by amplification of pLANTIGEN4 with oligonucleotides LTBsal, 5′-gagtcgtcgacacctggagcgtagttcttcatgc-3′ and LTBnco, 5′-gtgacgaagacaacatgaacaaggtgaagtgttatgt-3′ using a proof reading Pfu polymerase under optimum conditions. BpiI/SalI digested PCR fragment comprising the synthetic gene for LT-B, was cloned in NcoI/SalI digested binary vector pLANTIGEN25 and the resulting plasmid was cut with SalI and BamHI. A XhoI/BamHI fragment of pLANTIGEN25 comprising the sequence for SVCV G was ligated in the former digested plasmid resulting in pLANTIGEN27 comprising an LT-B-SVCV G fusion (FIG. 9). Solanum tuberosum Desiree was transformed with a 1:1 mix of pLANTIGEN4 and pLANTIGEN27 (co-transformation) using Agrobacterium tumefaciens-mediated transformation. 21 independent transgenic plants were generated for pLANTIGEN (4+27) and grown to maturity in the greenhouse.

Analysis of expression. Tuber extracts of pLANTIGEN25 tubers were analysed for the presence of SVCV G protein by standard ELISA or sandwich ELISA using various combinations of monoclonal antibodies G3C7, 4C12, 2C1/3H1, 2C1/3G2, 2C1/3C9, 2C1/A10/2G2, 2C1/A10/1H11 and 2C11/A10/D12 all raised against SVCV CZ539 strain and specific for G protein. None of the 47 plants exhibited expression of SVCV G. Analysis of pL(4+27) tuber extracts was initially performed by GM1 ELISA. Results are summarized in FIG. 10 and indicate that a number of transgenic plants show expression of GM1 Binding LT-B5 pentamers up to 4 micrograms/g FW tuber. To analyse the presence of SVCV G protein, a modified GM1 ELISA was performed in which the detection of bound complexes with VD12 (specific for LT-B5) was now done with monoclonal antibody 2C1/3C9 specific for SVCV G. Extracts from pL(4+27) plants that reacted positively in this modified GM1 ELISA are indicated in FIG. 10 with an arrow. As can be seen, most of the plants that were positive for GM1 binding LT-B5 also reacted with 2C1/3C9 showing the presence of SVCV G protein in the complex.

Immunogenicity and challenge experiments. Extracts and freeze dried tuber material of selected plants including pL(4+27)-11, 15, 17 and 23 will be used in a binding and uptake experiment with carp and vaccination trial and challenge experiment. A mix of freeze-dried tuber material comprising pL(4+27)-11, 15, 17 and 23 was incorporated into a standard fish feed to final concentration 20% tuber material as described in EXAMPLE 5. The resulting feed will be used for oral immunization of carp as described. Carps reared at 10° C. will be challenged 1, 12 and 21 weeks post vaccination by intraperitoneal injection of 0.2 ml of SVCV virus strain 539.

### EXAMPLE 5

### Oral Immunization of Carp

Gene constructs and transformation. Design and construction of the pLANTIGEN4 synthetic plant-optimized gene for expression of LTB in potato tuber and control PAT4, have been described previously (Lauterslager at al., 2001). pLANTIGEN15 was constructed by cloning two synthetic influenza virus hemagglutinin (HA) heavy chain decapeptide sequences, representing amino acids 111-120 of PR8HA-1 (Hackett et al., 1985), together with two synthetic sequences coding for the amino-terminal region of the viral VP2 protein of canine parvovirus (CPV; Langeveld et al., 1994) into the unique BamHI site of pLANTIGEN4. Each of the four sequences was cloned in such a way that they were separated by two alanine residues for spacing (LT-B-iipp). pLANTIGEN20 was constructed by cloning a sequence for green fluorescent protein (GFP) in frame into the unique BamHI site of pLANTIGEN4 as described in Example 3. Transformation of Solanum tuberosum cultivar Désirée, growth of transgenic plants and tuberisation are as described (Lauterslager et al., 2001).

Tuber protein and fish feed preparations. One transgenic line for pLANTIGEN 15 (pL1516) was selected based on a combination of good tuber setting and recLT-B production as estimated by GM1-ELISA. Identification and analysis of pL2003 harboring an LT-B-GFP fusion protein was described in Example 3. PAT4 control line comprising an empty vector cassette was described before (Lauterslager et al., 2001). All, pL1516, PAT4 and pL2003, were multiplied through seed tubers and grown in the greenhouse for bulk tuber production. Bulk tubers were harvested, peeled, cut into slices, freeze dried and homogenised by grinding using a mortar and pestle. Ground and homogenized material was used in immunization experiments with carp and for manufacturing a fish feed for oral immunizations. Incorporation into fish feed pellets was done by mixing 20% (final concentration) of pL2003 homogenized freeze-dried potato tuber material (approximately 21 micrograms of LT-B-GFP per gram dry weight tuber) with normal fish feed compounds prior to making the pellets. The resulting mix containing the potato material was converted into pellets and coated with fish oil according to standard methods. The feed was dried at room temperature before use. Final concentration of LT-B-GFP in feed was estimated 4.3 micrograms/g feed.

Carp binding and uptake experiments. Six month old carp (Cyprinus carpio L.) weighing around 20 g, were reared in re-circulating, filtered and UV-sterilised water at 3° C. The fish were fed with standard food pellets (Skretting/Nutreco, Putten, The Netherlands) at a daily ration of 2.5% of their body weight. Twenty-four hours prior to an intubation or in feed immunization experiment, the fish were fasted. pL2003 homogenized freeze-dried tuber material or control PAT4 were resuspended in PBS to approximately 0.4 μg LT-B-GFP/100 μl suspension (pL2003 suspension). recGFP purified from E. coli extracts was diluted in PBS to final concentration 13.5 μg/100 μl. Groups of 3 fish were anally intubated with 100 μl recGFP suspension (13.5 μg), pL2003 suspension (approximately 0.4 μg LT-B-GFP) or PAT4. Six hours post intubation the fish were killed by an overdose of Tricaine Methanesulfonate (TMS, Crescent Research Chemicals, AZ) and the end gut was removed, snap frozen in liquid nitrogen and stored at −80° C. prior to analysis. Gut tissue was cut using a cryostat (Reichert-jung 2800 Frigocut N, Nussloch, Germany) and tissue sections were embedded in vectashield (Vector Laboratories Inc., Burlingame, Calif.), containing propidium iodide. Gut section analysis by laser scan microscopy (Zeiss LSM-510, Jena, Germany) clearly showed that LT-B-GFP (pL2003 material) is taken up more efficiently compared to recGFP (FIG. 11). GFP could be detected in the supranuclear vacuoles of enterocytes and also in macrophage-like cells underlying the epithelium suggesting transport from enterocytes into large macrophage-like cells (FIG. 11B). Although little GFP could be detected in enterocytes of fish intubated with GFP only, the signal was less intense compared to the LT-B-GFP treated fish and virtually no GFP could be detected in the macrophage-like cells (FIG. 11A). No signal was detected in gut of carp intubated with control potato PAT4 material, showing that although the concentration of the applied GFP was higher than the potato derived LT-B-GFP, its uptake is far less efficient suggesting an additive effect of LT-B.

Immunization of carp. Potato tubers expressing LT-B-iipp (pL1516; approximately 5.3 micrograms of LT-B-iipp/g FW) and PAT4 were freeze dried, ground, homogenized and resuspended in PBS. Estimated amount of LT-B-iipp in freeze-dried pL1516 material is 21.2 micrograms/g DW. Fish (3 per group) were intubated with 300 μl potato pL1516 suspension (approximately 3 μg LT-B-iipp) One group of fish was killed 3 weeks post intubation and blood was collected for measurement primary response. A second group of fish was boostered anally after 8 weeks (secondary response) and killed 2 weeks post boostering, upon which blood samples were taken. Blood samples were allowed to cloth for 18 hours at 4° C., were centrifuged (10000×g, 5 min. RT) and serum was collected.

Immune responses were measured as follows: Maxisorb ELISA plates (Nunc, Roskilde, Denmark) were coated for 18 hr at 4° C. with 100 ml of 4 mg/ml anti-parvo peptide monoclonal antibody (3C9, Ingenaza, Madrid, Spain) followed by blocking with 0.5% BSA (Roche, Mannheim, Germany) for two hours at RT. Subsequently a synthetic peptide representing the amino-terminal region of the viral VP2 protein of canine parvovirus (CPV; Langeveld et al., 1994) was added. Sera were added and serially diluted. Detection was with biotin linked monoclonal antibody recognising carp serum Ig (WCI-12). Samples were diluted in PBS containing 0.5% BSA and 0.1% Tween 20 (Merck) and bound WCI-12 was visualized using streptavidin linked HRP (Sanquin, Amsterdam, The Netherlands, dilution: 1:5,000) and TMB peroxidase substrate (Kirkegaard & Perry, Gaithersburg, Md.). The substrate was incubated for a maximum of 20 min, and subsequently the OD was measured at 450 nm. Anti parvo titres (pL1516 immunized fish) were defined by the dilution of the sample at an OD of 0.1. Anti-parvo responses could be detected in both primary and secondary immune serum by ELISA and results are summarized in FIG. 12. FIG. 12 shows that the titres measured in the sera of fish in the pL1516 (LT-B-iipp) treated group is on average higher compared to the control group showing that upon anal intubation antigen specific systemic humoral immune responses are induced.

In another experiment, carp were fed during 5 consecutive days with the pL2003 containing feed and PAT4 at a daily ration of 6% of their body weight. Four weeks after the last immunisation, sera were isolated and treated as described above (primary response). Eight weeks after the last immunisation fish were boostered by feeding them for one day at a ration of 4% of their body weight. Four weeks after the booster serum was isolated and treated as described above. As a positive control carp were both primed and boostered with 50 μg of purified recGFP in incomplete freunds adjuvant by intraperitoneal injection. For detection of anti-GFP immune responses, Maxisorb ELISA plates (Nunc) were coated for 18 hr at 4° C. with 100 ml of 0.5 mg/ml anti-GFP antibody (ab1218, Abcam, Cambridge, UK) followed by blocking with 0.5% BSA (Roche) for two hours at RT. Subsequently 100 μl recGFP (1 mg/ml) was added and sera were added at serial dilutions. Anti-GFP specific serum antibodies were detected using biotin linked mAb recognising carp serum Ig (WCI-12). Samples were diluted in PBS containing 0.5% BSA and 0.1% Tween 20 (Merck). Finally WCI-12 was detected using streptavidin linked HRP (Sanquin, Amsterdam, The Netherlands, dilution: 1:5,000) and TMB peroxidase substrate (Kirkegaard & Perry, Gaithersburg, Md.). The substrate was incubated for a maximum of 20 min, and subsequently the OD was measured at 450 nm. Anti GFP titres were defined by the dilution of the sample at an OD of 0.05 (primary response) or 0.2 (secondary response). Results are summarized in FIG. 13 and clearly show that in secondary immune sera of carp fed with the pL2003 containing feed pellets, an elevated anti-GFP titre was detected (FIG. 13) whereas control pellets did not evoke a GFP specific humoral immune response. These data show that oral administration of LT-B-GFP evokes an systemic humoral immune response.

### REFERENCES

Aitken, R., Hirst, T. R. (1993) Recombinant enterotoxins as vaccines against Escherichia coli-mediated diarrhea. Vaccine 11: 227-233.
Cardenas, L., Clements, J. D. (1993) Development of mucosal protection against the heat-stable enterotoxin (ST) of Escherichia coli by oral immunization with a genetic fusion delivered by a bacterial vector. Infection and Immunity 61: 4629-4636.
Dalmo, R. A., Ingebrigtsen, K., Bgwald, J. (1997) Non-specific defense mechanisms in fish, with particular reference to the reticuloendothelial system (RES). Journal of Fish Diseases 20: 241-273.
De Haan L, Verweij W R, Feil I K, Holtrop M, Hol W G, Agsteribbe E, et al. Role of GM1 binding in the mucosal immunogenicity and adjuvant activity of the Escherichia coli heat-labile enterotoxin and its B subunit. Immunology 1998; 94(3):424-30.
Hart S., Wrathmell A. B., Harris J. E. and Grayson T. H. (1988) Gut immunology in fish: a review. Developmental and Comparative Immunology 12: 453-480.
Jagusztyn-Krynicka, E. K., Clark-Curtiss, J. E., Curtiss III, R. (1993) Escherichia coli heat-labile toxin subunit B fusions with Streptococcus sobrinus antigens expressed by Salmonella typhimurium oral vaccine strains: importance of the linker for antigenicity and biological activities of the hybrid proteins. Infection and Immunity 61: 1004-1015.
Khoury, C. A., Meinersmann, R. J. (1995) A genetic hybrid of the Campylobacter jejuni flaA gene with LT-B of Escherichia coli and assessment of the efficacy of the hybrid protein as an oral chicken vaccine. Avian Diseases 39: 812-820.
Lamers C. H. J., (1985) The reaction of the immune system of fish to vaccination. PhD thesis, Agricultural University of Wageningen.
Leong, J. C., Fryer, J. L. (1993) Viral vaccines for aquaculture. Annual Review of Fish Diseases 3: 225-240.
Lorenzen, N., Olesen, N. J. (1997) Immunisation with viral antigens: viral haemorrhagic septicaemia. In: Fish Vaccinology. Gudding, R., Lillehaug, A., Midlyng, P. J., Brown, F. (eds). Dev. Biol. Stand. Basel, Karger, vol. 90, p 201-209.
Lorenzen, N., Olesen, N. J., Koch, C. (1999) Immunity to VHS virus in rainbow trout. Aqua-culture 172: 41-61.
Mowat, A. M. (1994) Oral tolerance and regulation of immunity to dietary antigens. In: Handbook of mucosal immunology (Ogra, P. C., Lamm, M. E., Mestecky, J., Strober, W., McGhee, J. R., Bienenstock, J., eds.) pp. 185-201. San Diego: Academic Press.
Newman, S. G. (1993) Bacterial vaccines for fish. Annual Reviews Fish Diseases 3: 145-185.
Rombout J. H. W. M., Lamers C. H. J., Helfrich M. H. Dekker A. and Taverne-Thiele J. J. (1985) Uptake and transport of intact macromolecules in the intestinal epithelium of carp (Cyprinus carpio L.). Cell Tissue Research 239: 519-530.
Rombout, J. H. W. M., van den Berg A. A. (1989) Immunological importance of the second gut segment of carp. I. Uptake and processing of antigens by epithelial and macrophages. Journal of Fish Biology 35: 13-22.
Rombout, J. H. W. M., van den Berg A. A., van den Berg, C. T. G. A., Witte, P., Egberts, E. (1989) Immunological importance of the second gut segment of carp. III. Systemic and/or mucosal immune responses after immunization with soluble or particulate antigen. Journal of Fish Biology 35: 179-186.
Walker, R. I. (1994) New strategies for using mucosal vaccination to achieve more effective immunization. Vaccine 12: 387-400.
Weiner, H. L., Friedman, A., Miller, A., Khoury, S. J., Al-Sabbagh, A., Santos, L., Sayegh, M., Nussenblatt, R. B., Trentham, D. E., Hafler, D. A. (1994) Oral tolerance: Immunologic mechanisms and treatment of animal and human organ-specific autoimmune diseases by oral administration of autoantigens. Annual Review in Immunology 12: 809-837.

1. A method for the expression of a protein of interest in a host cell, comprising providing said host cell with a recombinant nucleic acid construct encoding a fusion protein comprising said protein of interest fused to the B subunit of a member of the AB5-class of bacterial toxins, and wherein said protein of interest originates from an organism that has an optimal growth temperature that lies below the optimal growth temperature of said host cell.

2. A method according to claim 1, wherein said B subunit is the B-subunit of Vibrio cholerae cholera CT toxin (CT-B) or Escherichia coli heat-labile LT enterotoxin (LT-B).

3. A method according to claim 1, wherein said host cell is an edible host cell.

4. A method according to claim 1, wherein said host cell is a plant host cell, preferably a host cell of a tuberous plant.

5. A method according to claim 1, wherein said protein of interest is an antigen.

6. A method according to claim 1, wherein said protein of interest originates from a virus or micro-organism pathogenic to fish.

7. A method according to claim 6, wherein said virus or micro-organism pathogenic to fish is selected from the group consisting of infectious pancreatic necrosis virus (IPNV), striped jack nervous necrosis virus (SJNNV), infectious haematopoietic necrosis virus (IHNV), viral haemorrhagic septicaemia virus (VHSV), Pancreas Disease virus (SPDV), infectious salmon anaemia virus (ISAV), Spring Viraemia of Carp virus (SVCV), Koi Herpesvirus (KHV), Flexibacter columnaris, Edwardsialla ictaluri, E. tarda, Piscirickettsia salmonis, Vibrio spp and Aeromonas spp., Yersinia ruckeri, Pasturella piscicida and Renibacterium salmoninarum.

8. A method according to claim 7, wherein said antigen of interest is selected from the group consisting of the VP2-protein of IPNV, the glycoprotein of VHSV (VHSV-G) and the glycoprotein of SVCV (SVCV-G).

9. A method according to claim 1, wherein said B-subunit is fused to the N-terminus of said protein of interest.

10. A fusion protein obtainable by a method according to claim 1.

11. A protein complex comprising a fusion protein according to claim 10.

12. A vaccine composition comprising a fusion protein of claim 10.

13. Vaccine composition of claim 12, being an oral fish vaccine composition.

14. A method for immunizing fish, comprising the oral administration of a vaccine composition of claim 13 to a fish.

15. Fish feed comprising a fusion protein of claim 10.

16. Use of a fusion protein of claim 10 for the manufacture of an oral fish vaccine or a fish feed.

17. A nucleic acid construct for use in a method of claim 1, encoding a fusion protein comprising a protein of interest which originates from a virus or micro-organism pathogenic to fish, fused to the B subunit of a member of the AB5-class of bacterial toxins, preferably the B-subunit of Vibrio cholerae cholera toxin (CT-B) or Escherichia coli heat-labile enterotoxin (LT-B).

18. Nucleic acid construct of claim 17, wherein said virus or micro-organism pathogenic to fish is selected from the group consisting of infectious pancreatic necrosis virus (IPNV), striped jack nervous necrosis virus (SJNNV), infectious haematopoietic necrosis virus (IHNV), viral haemorrhagic septicaemia virus (VHSV), Pancreas Disease virus (SPDV), infectious salmon anaemia virus (ISAV), Spring Viraemia of Carp virus (SVCV), Koi Herpesvirus (KHV), Flexibacter columnaris, Edwardsialla ictaluri, E. tarda, Piscirickettsia salmonis, Vibrio spp and Aeromonas spp., Yersinia ruckeri, Pasturella piscicida and Renibacterium salmoninarum.

19. Nucleic acid construct of claim 18, wherein said protein of interest is an antigen selected from the group consisting of the VP2-protein of IPNV, the glycoprotein of VHSV (VHSV-G) and the glycoprotein of SVCV (SVCV-G).

20. Expression vector, preferably a plant expression vector, comprising a nucleic acid construct according to claim 17.

21. A vaccine composition comprising a protein complex of claim 11.

22. A vaccine composition of claim 21, being an oral fish vaccine composition.

23. Fish feed comprising a protein complex of claim 11.

24. Use of a protein complex of claim 11 for the manufacture of an oral fish vaccine or a fish feed.
